(19) United States
(12) Patent Application Publication
(10) Pub. No.: US 2010/0112055 A1
(43) Pub. Date: May 6, 2010

(54) MICROPOROUS FILM AND PREPARATION AND USE THEREOF

(75) Inventor: WEN-JEN LIN, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: NATIONAL TAIWAN UNIVERSITY, Taipei City (TW)

(21) Appl. No.: 12/405,509

(22) Filed: Mar. 17, 2009

(30) Foreign Application Priority Data
Oct. 30, 2008 (TW) 097141795

(51) Int. Cl. A61K 9/24; A61K 47/38; A61K 47/30

(52) U.S. Cl. 424/473; 514/781; 514/772

(57) ABSTRACT

The present invention is a new type of microporous film. The micoporous film can be applied to use as coating material of controlling drug release. The present invention also relates to a preparation of the microporous film.

### FIELD OF THE INVENTION

The present invention relates to a microporous film for use as coating material of controlling drug realease and could be applied to a solid dosage form such as tablet for controlling drug release.

### BACKGROUND OF THE INVENTION

The osmotic-controlled dosage form is an oral sustained release system, which comprises three basic major components: (1) a semipermeable polymeric film of the outermost layer; (2) a drug core in the center; (3) and a single orifice drilled by laser on the semipereable polyemeric film. The semipermeable membrane allows water to enter the drug core to disintegrate it, but the dissolved drug can not release freely through the membrane. Therefore, the single orifice drilled by laser produces the pathway for drug release. The disadvantage resided in that orifice is easily blocked by the incomplete dissolved drug particle, so as to affect the following drug release. Moreover, the laser drilling technique belongs to high technology and the cost is expensive.

Republic of China Patent No. 00477802 discloses “A method for preparing hydrophilic porous polymeric materials”, which comprises the step of uniform mixing a hydrophilic polymeric material (for example, a natural hydrophilic protein or a polysaccharide polymer and combined material thereof) with a hydrophobic material; solvent sintering the surface of the hydrophilic polymeric material with water or aqueous solution; removing the hydrophobic material contained within the hydrophilic polymeric matrial with a massive organic solvent to produce high porosity of the porous hydrophilic polymer material.

U.S. Pat. No. 5,827,538 discloses “Osmotic devices having vapor-permeable coatings” providing a permeable membrane, which uses osmagent (such as sugar, polyethylene glycol, or sodium lauryl sulfate, etc.) to generate osmotic pressure to control water penetrating the lipophilic coating membrane (such as polyethylene or poly(vinylidene difluoride)). In other words, water cannot penetrate the membrane when the osmotic pressure is less than the threshold; when the osmotic pressure generated by the osmagent is greater than the threshold, it begins to open the porous structure of the lipophilic membrane and to allow water to penetrate the membrane. Therefore, the scope of the patent focus on the composition of osmagent to active and control water penetrating the membrane.

### SUMMARY OF THE INVENTION

The present invention provides a microporous film comprising:
(a) a semipermeable polymer; and (b) a water-soluble polymer, wherein polymers (a) and
(b) form a uniform-blending state through a solvent.

The present invention also provides a method for preparing a microporous film comprising the steps of:
(a) choosing a suitable formula consisting of a semipermeable polymer, a water-soluble polymer and a solvent;
(b) adding the solvent to completely dissolve the semipermeable polymer and the water-soluble polymer to form a polymer blended solution;
(c) controlling temperature of the polymer blended solution and volatile speed of the solvent; and
(d) forming the film when the solvent is evaporated completely.

The present invention further provides a method of preparing a micropore-controlled release tablet, which comprising: producing a drug core tablet with drug and excipient; preheating the drug core tablet; coating the drug core tablet with a polymer blended solution, wherein the polymer blended solution comprising: (a) a semipermeable polymer; and (b) a water-soluble polymer, wherein the polymers (a) and (b) form a uniform-blending state through a solvent.

### BRIEF DESCRIPTION OF THE DRAWINGS

FIG. 1 shows the flow chart of making a microporous film.

FIG. 2 shows the flow chart of preparing micropore-controlled release tablets.

FIG. 3 illustrates the proposed model for micropore-controlled release tablets.

FIG. 4 is the scanning electron microscope (SEM) micrograph of surface morphology of microporous film of the present invention.

FIG. 5 is the SEM micrograph of vertical cross section morphology of microporous film.

FIG. 6 shows the release of theophylline from tablets coated by various ratios of (a) CA/PEG₄₀₀₀ and (b) CA/PEG₁₀₀₀₀.

FIG. 7 shows the effect of adding different types of excipients on (a) hardness, (b) disintegration time, (c) theophylline solubility, and (d) osmotic pressure of drug core tablets.

FIG. 8 shows the release of of theophylline from CA_{50%}/PEG_{50%} micropore-controlled release tablets in the absence (TH-A₅₀) and presence of different types of excipients.

FIG. 9 shows the release of theophylline from micropore-controlled release tablets in the absence or presence of 50% lactose.

FIG. 10 shows the plasma drug concentrations after intravenous injection of theophylline solution or oral administration of uncoated tablet (TH-Lac) and coated tablet (TH-Lac-A₅₀), respectively.

FIG. 11 shows the correlation between the percentage of drug absorbed in vivo and the percentage of drug released in vitro.

### DETAILED DESCRIPTION OF THE INVENTION

The present invention relates to develop a microporous film and a micropore-controlled release tablets. The key conditions need to be managed in the process of preparing the tablets are: the type of solvents used to dissolve polymer, the molecular weight and the amount of the water-soluble polymers, temperature and volatile speed of the solvent during the formation of a thin film, solubility of the drug core, different types of excipient and other factors. These factors mentioned above affect the formation integrity of films, the size and number of micropores, and drug release rate through the film. Thus, in light of the therapeutic dose and drug concentration essential for clinical use, the adequate compositions and the preparation conditions for the micropore-controlled release tablets can be selected in order to achieve the reasonable release duration.

The microporous film of this invention is designed by combination of two immiscible polymers. One of the polymers with a semipermeable characteristic forms the main body of the film, and another is a water-soluble polymer as a pore-forming agent. The water-souble polymer can be leached out from the film in the aqueous environment and formed many interconnected micropores. These micropores not only enhance water to continuously diffuse into drug core to dissolve the drug, but also allow dissolved drug to release out through these micropores. In particular, after water-soluble polymer leaching out forms microporous structure in the film which provides the main way for drug release.

U.S. Pat. No. 5,827,538, discloses the permeable membrane coating. The osmotic pressure generated by osmagent activates the micropore structure of lipophilic polymer to allow water penetration. The characteristic of this patent is the design of osmagent component to activate and control water penetration through film. Therefore, the present invention is different from the U.S. Patent technology.

The micropores on the film of the present invention have uniform size with good reproducibility and homogeneous distribution. The micropores not only provide the route for drug sustained release but also avoid using high technique laser to drill the orifice for drug release. In other words, it eliminates the defect of the single orifice blocked by the disintegrated drug particle which can obstruct drug release. In addition, the water-soluble polymer used in the present invention is widely used in pharmaceutical applications, and is high safety, low cost, easy purchase and has a variety of molecular weights for selection.

The present invention uses water-soluble polymers of molecular weight range from about several thousand to ten thousand as a pore-forming agent. There are a variety of molecular weights of water-soluble polymers available in the market. In Taiwan and the United States patents, it is common use of low molecular weight polymer (up to hundreds) as a plasticizer and high molecular weight polymer (up to millions) as a swelling agent, whereas there is no acted as a pore-forming agent. Using the water-soluble polymers as pore-froming agent not only avoids using laser drilling technique but also prevents blocking the single orifice for drug release. The microporous film of the present invention can also be used as coating materials for tablets, granules, microspheres, and other solid dosage forms to sustain and control drug release. The microporous film of the present invention can be widely applied in the pharmaceutical industry due to easy fabrication, and the prime cost is expected to be reduced.

Republic of China Patent No. 00477802 discloses “A method for preparing hydrophilic porous polymeric materials”. The materials used in the patent is different from those in the present invention, and the preparation method between the patent and the present invention is also distinct. The patent mainly uses natural hydrophilic proteins or carbohydrate polymers to produce porous polymeric material whereas the present invention uses a semipermeable polymer to form microporous film. Further, the patent discloses that the pores formed on the material after treatment of organic solvent. However, the water-soluble polymers used in the present invention can be directly dissolved in body fluid or water without using organic solvent. Therefore, the present invention is suitable for coating various solid dosage forms to control drug release in vivo.

The present invention provides a microporous film, which comprises: (a) a semipermeable polymer, and (b) a water-soluble polymer, wherein the polymers (a) and (b) form a uniform-blending state through a solvent.

The term “semipermeable polymer” used herein refers to the main body of the microporous film allowing water to diffuse freely. The semipermeable polymer includes but is not limited to cellulose acetate, methyl cellulose acetate (MCA), cellulose diacetate (CDA), cellulose triacetate (CTA). The semipermeable polymer in the microporous film of the present invention is from 50 to 95% by weight.

The term “water-soluble polymer” used herein refers to the polymer which is water-soluble. The water-soluble polymer includes but is not limited to polyethylene glycol, polypropylene glycol, and polyethylene-propylene glycol copolymer. The molecular weight can range from 1,000 to 20,000 daltons, preferably 4,000 to 10,000 daltons. In particular, the water-soluble polymer in the film can be leached out in an aqueous solution to form micropores, and its weight percentage is correlated with the density of the micropores. The water-soluble polymer in the microporous film of the present invention is from 5 to 50% by weight.

The term “solvent” used herein means a liquid which can dissolve two immiscible semipermeable polymer and water-soluble polymer to form a polymer blended solution. The solvent includes but is not limited to ketones (such as acetone), esters (such as ethyl acetate), alcohols (such as ethanol), alkanes (such as methylene chloride, dioxane), amides (such as dimethyl formamide), polar solvents (such as water) or a mixure of above. For example, ketone and ester solvent mixure is in volume ratio of 2:1 to 1:2, preferably mixture is ketone, ester and alcohol solvent mixture with the volume ratio of 6:6:1.

The present invention further provides a method of preparing a micorporous film, comprising the steps of: (a) chosing a suitable formula consisting of a semipermeable polymer (such as cellulose acetate), a water-soluble polymer (such as polyethylene glycol) and a solvent (such as acetone, ethyl acetate, ethanol or a combination of above), (b) adding solvent to completely dissolve the semipermeable polymer and the water-soluble polymer to form a polymer blended solution, (c) controlling temperature of the polymer blended solution and volatile speed of the solvent, and (d) forming the pre-microporous film when the solvent is volatiled completely.

The consideration of choosing the suitable formula consisting of semipermeable polymer, water-soluble polymer, and solvent is: (a) the molecular weight of the water-soluble polymer (4,000 or 10,000), (b) the content ratio of the semipermeable polymer and the water-soluble polymer (the water-soluble polymer is from 5 to 50% by weight, and the semipermeable polymer is from 50 to 95% by weight), (c) the type of solvent to dissolve the polymers, (d) the concentration of the polymer blended solution.

The present invention can further place the pre-microporous film into water to leach out the water-soluble polymer to form a microporous film.

The present invention also provides a method for preparing micropore-controlled release tablets, which comprises: preparing drug core by using drug and excipient; preheating the drug core; coating the drug core with a polymer blended solution many times wherein the polymer blended solution can form a pre-microporous film on the drug core, and the pre-microporous film comprising: (a) a semipermeable polymer; and (b) a water-soluble polymer, wherein the two polymers (a) and (b) form a uniform-blending state through a solvent.

The term “excipient” used herein means ingredients other than the essential components of drug, including disintegrant, binder, surfactant, buffer, flavor agent, antioxidant, preservative, and coloring agent. The preferable excipient includes but is not limited to lactose, potassium chloride, corn starch, polyvinylpyrrolidone K30 and polyvinylpyrrolidone K90, and the more preferable excipient is lactose, which accounts for 20-50%, preferable 20% by weight based on the total weight of the drug core.

The term “drug” used herein refers to any kinds of drugs, preferably is drug powder, and can be compressed into drug core with excipient. In the preferred embodiment, the drug is selected from xanthine or its derivatives such as theophylline, diprophylline, proxyphylline, theobromine, aminophylline; the more preferable drug is theophylline.

The drug powder and various types of excipients are sieved and mixed in geometric dilution method, then sieved again and prepared the drug core using direct compression method. The product of oral controlled relaease tablet can further dissolve the water-soluble polymer in vivo to form the micropore-controlled release tablet, which allows water to diffuse through the semipermeable polymer or the micropore channels into the drug core, and the dissolved drug can be released from the micropores. The weight percentage of water-soluble polymer is positive correlated with the density of micropores, which can affect the drug release rate and release time resulting in sustained release of drugs up to 18 to 36 hours.

In the method of the present invention, the drug core was preheated so as to facilitate follow-up coating by polymer blended solution via dip-coating method. Preheating temperature is depended on the thermal property of drug and the characteristics of the film. The embodiment (such as theophylline as the drug core) is 25 to 75° C. (the preferable temperature is 30 to 60° C.), and heated 1 to 10 minutes (the preferable heating time is 3 to 7 minutes).

### EXAMPLE 1

Preparation of Polymer Blended Solutions:

Both semipermeable polymer like cellulose acetate and water-soluble polymer like polyethylene glycol (PEG₄₀₀₀ or PEG₁₀₀₀₀) were previously weighed (0, 5, 10, 20, 30, 40, and 50% w/w of the water-soluble polymer) and dissolved in the blended solvents of acetone, ethyl acetate, and ethanol with a certain volume ratio of 6:6:1. Shake the polymer blended solution until the polymers were completely dissolved to form a certain concentration of coating solution.

Preparation of Microporous Film

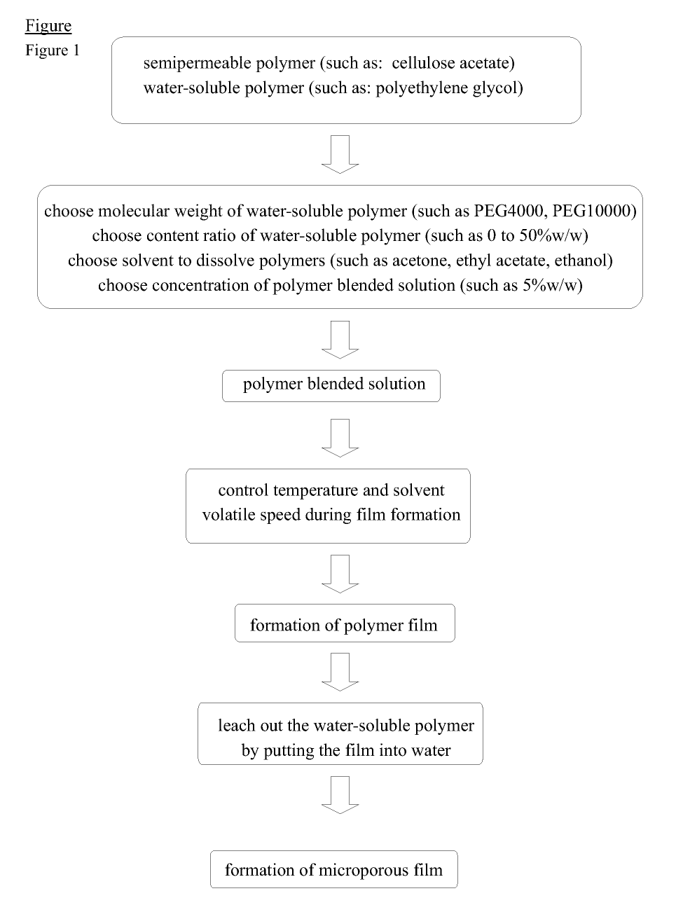

A certain volumn of the aforementioned polymer blended solution was put on a glass disk and dried in a vacuum oven. The pre-microporous film was fromed when the solvent was volatiled. Film was immersed in de-ionized water several days and the water was changed many times to ensure that the water-soluble polymer had been completely washed out. The film was then put into a vacuum oven to remove residual moisture and the microporous film was obtained (see FIG. 1).

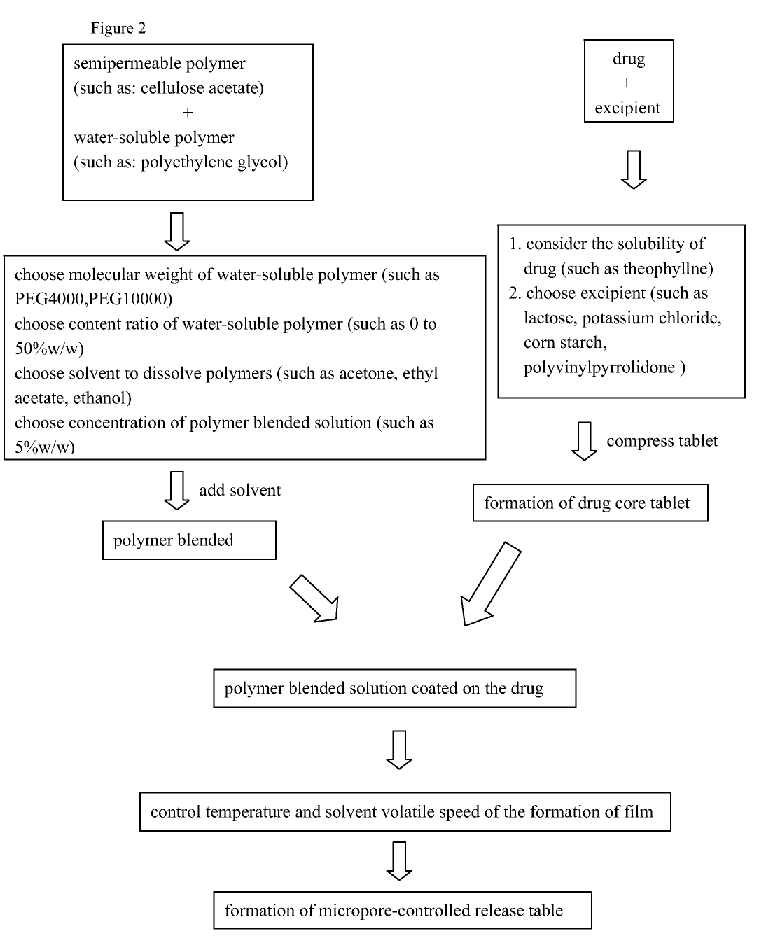

The flow chart of preparing micropore-controlled release tablets was shown in FIG. 2, comprising: (a) a drug core tablet; and (b) the outer microporous film.

Preparation of Drug Core Tablet

Drug powder (such as theophylline) and a variety of excipients (such as lactose, potassium chloride, corn starch, polyvinylpyrrolidone K30 and polyvinylpyrrolidone K90) were sieved over 200-mesh sieve, respectively, then mixed the above drug powder and excipient by geometric dilution method, and sieved over another 120-mesh sieve to remove the aggregation powder. The seieved powder mixture was weighted and put into a 3-mm diameter of die, and compressed under 2,200 pounds force for 10 seconds to produce the drug core tablet. The hardness and disintegration time of drug core tablet were measured. The solubility of theophylline in the presence of different types of excipients was investigated at 37° C. under continuous shaking. The solution was filtered through a 0.45-μm filter, and the concentration of theophylline was measured by UV spectrophotometer at 272 nm after proper dilution. The osmotic pressure of filtrate was measured by osmometer.

Preparation of Micropore-Controlled Release Tablets

The drug core was preheated 3 to 7 minutes under 30 to 60° C. The polymer blended solution was coated on each drug core several times via a dip-coating method, and the coated tablets were dried in the oven until the solvent was volatiled completely. Finally, the theophylline coated tablets with various compositions of CA/PEG₄₀₀₀ and CA/PEG₁₀₀₀₀ were obtained. The composition and symbol for theophylline coated tablets are listed in Table 1.

TABLE 1
The film and core compositions for a series of
micropore-controlled release tablets

| | Film | Core composition | | |
|---|---|---|---|---|
| | composition | | Excipient | |
| Symbol | CA/PEG | Theophylline | | weight |
| Coated Tablet | (% w/w) | (mg) | type | (mg) |
| TH | — | 30 | — | |
| TH-A₀* | 100:0 | 30 | — | |
| TH-A₅* | 95:5 | 30 | — | |
| TH-A₁₀* | 90:10 | 30 | — | |
| TH-A₂₀* | 80:20 | 30 | — | |
| TH-A₃₀* | 70:30 | 30 | — | |
| TH-A₄₀* | 60:40 | 30 | — | |
| TH-A₅₀* | 50:50 | 30 | — | |
| TH-B₀** | 100:0 | 30 | — | |
| TH-B₅** | 95:5 | 30 | — | |
| TH-B₁₀** | 90:10 | 30 | — | |
| TH-B₂₀** | 80:20 | 30 | — | |
| TH-B₃₀** | 70:30 | 30 | — | |
| TH-B₄₀** | 60:40 | 30 | — | |
| TH-B₅₀** | 50:50 | 30 | — | |
| TH-Sta-A₅₀* | 50:50 | 24 | Starch | 6 |
| TH-KCl-A₅₀* | 50:50 | 24 | KCl | 6 |
| TH-Lac-A₅₀* | 50:50 | 24 | Lactose | 6 |
| TH-K30-A₅₀* | 50:50 | 24 | Kollindon ® 30 | 6 |
| TH-K90-A₅₀* | 50:50 | 24 | Kollindon ® 90 | 6 |
| TH-Lac₅₀ | — | 15 | Lactose | 15 |
| TH-Lac₅₀-A₅₀* | 50:50 | 15 | Lactose | 15 |
| TH-Lac₅₀- | 50:50 | 15 | Lactose | 15 |
| B₅₀** | | | | |

*A represents tablets coated by CA/PEG₄₀₀₀
**B represents tablets coated by CA/PEG₁₀₀₀₀

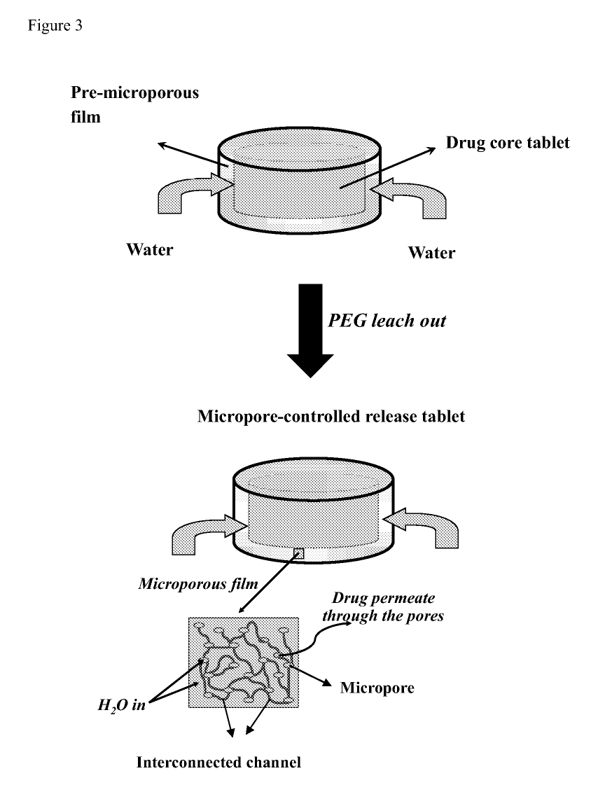

The model for drug release from micropore-controlled release tablet was proposed in FIG. 3. Semipermeable polymer allowed water to penetrate into the drug core at beginning. Simultaneously, the pore-forming agent PEG started to leach out of the coating film in water, and the interconnected micropores were formed in the film. These micropores not only enhanced water diffusion into drug core to dissolve drug, but also provided the route for drug release out of the micropore-controlled release tablets. In other words, the water-soluble polymers leached out from the film to form micropores providing the main route for drug release. The present invention can control drug release rate by changing the density of micropores.

Morphology of Microporous Film

The microporous film was observed using scanning electron microscope.

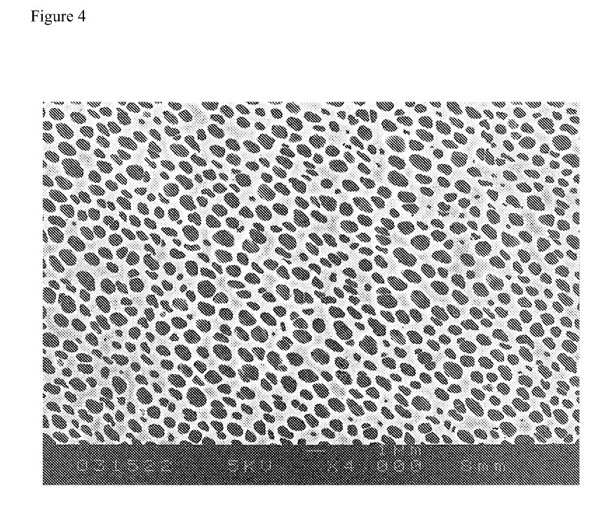

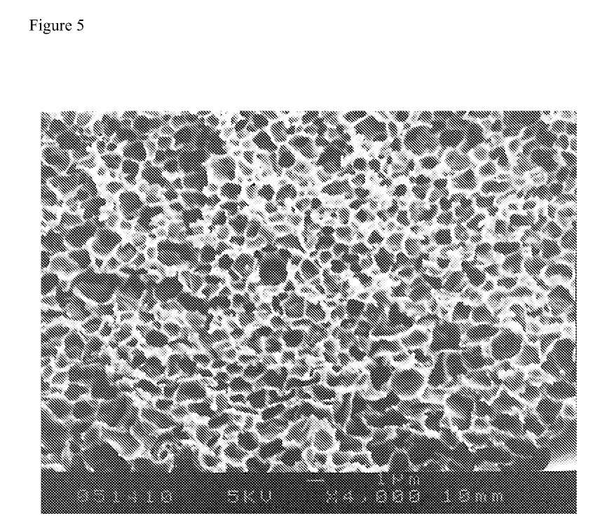

FIGS. 4 and 5 showed the surface and cross section morphology of microporous films, respectively. The coating film was composed by a semipermeable polymer cellulose acetate and a water-soluble polymer polyethylene glycol as a pore-forming agent. Adequate amount of cellulose acetate and polyethylene glycol (0 to 50% w/w) were dissolved in the blended solvents of acetone, ethyl acetate, and ethanol with a volume ratio of 6:6:1. After the solvent was removed, the formed coating film was further immersed into water for 36 hours to form microprous film.

### EXAMPLE 2

In Vitro Release Study

The dissolution of uncoated theophylline core tablets and the release of theophylline from coated tablets were conducted according to the USP basket method. De-ionized water was used as the dissolution medium and maintained at 37±0.5° C. The stirring speed was set at 50 rpm. Samples (1 mL) were withdrawn at specific time points, and the same volume of fresh medium was replaced. The concentration of theophylline in each sample was determined by validated UV spectrophotometer at 272 nm. In all cases three runs were carried out for each formulation. The release of drug from coated tablets was further fitted by equation (1):

$$[ \frac{{M}_{t}}{{M}_{∞}} ] = {Kt}^{n} \qquad ( 1 )$$

Mₜ: the amount of drug released at time t

M_{∞}: total amount of drug in each tablet

K: the release rate constant

n: the exponent constant

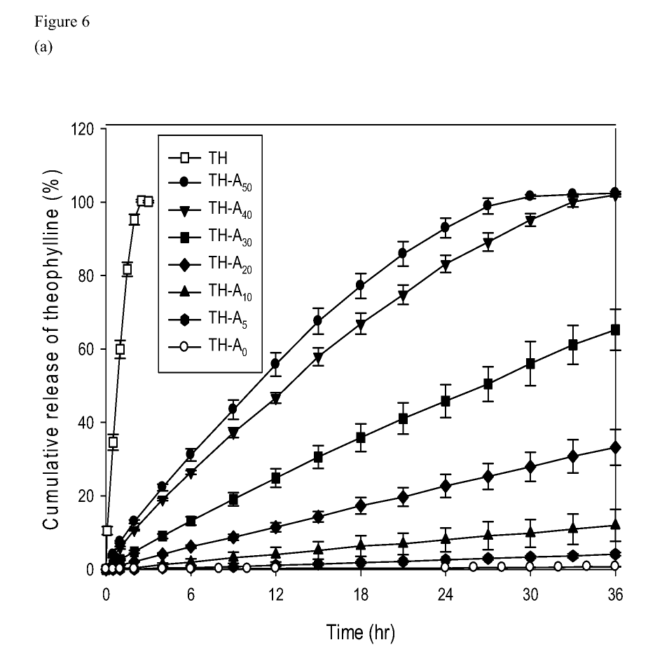

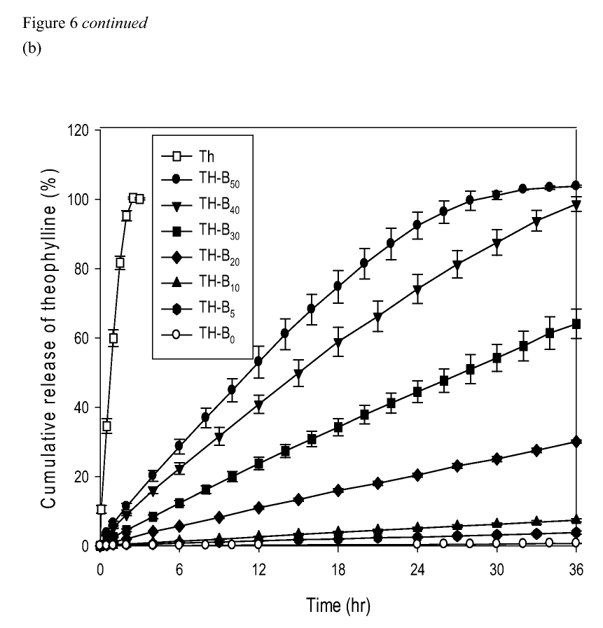

FIG. 6 showed the cumulative release of theophylline from micropore-controlled release tablets coated by various compositions of (a) CA/PEG₄₀₀₀ and (b) CA/PEG₁₀₀₀₀. There were less than 1% of drug released from 100% CA-coated tablet (TH-A₀) within 36 hours, and this result implied that theophylline cannot directly diffuse through CA. The uncoated theophylline tablet (TH) was completely dissolved in the release medium within 2.5 hours, however, a sustained-release character was observed for CA/PEG coated tablets. Increase in the level of PEG from 5% to 50% in the blended films prominently enhanced theophylline release. This result revealed that the release of theophylline from micropore-controlled release tablets was dominated by the density of micropores after PEG leaching out. Both CA/PEG₄₀₀₀ coated tablets and CA/PEG₁₀₀₀₀ coated tablets showed similar release pattern irrespective of the molecular weight of pore-forming agent. The release of theophylline from CA/PEG micropore-controlled release tablets was critically dominated by the micropores on the CA films. Since the microporous films formed by the same level of PEG₄₀₀₀ and PEG₁₀₀₀₀ had similar pore size and pore density, this results in the similar release profiles. The release rate constants K and n values were listed in Table 2. The release rate constants of coated tablets were smaller than uncoated tablets by 5-500 folds dependent of the level of PEG but irrespective of the molecular weight of PEG.

TABLE 2
The K and n values related to theophylline released from CA/PEG coated tablets

| CA/PEG₄₀₀₀ | K | | CA/PEG₁₀₀₀₀ | K | |
|---|---|---|---|---|---|
| Coated Tablet | (% h⁻ⁿ) | n | Coated Tablet | (% h⁻ⁿ) | n |
| TH-A₅ | 0.07 ± 0.01 | 1.16 ± 0.04 | TH-B₅ | 0.17 ± 0.01 | 0.86 ± 0.01 |
| TH-A₁₀ | 0.32 ± 0.18 | 1.03 ± 0.04 | TH-B₁₀ | 0.23 ± 0.02 | 0.97 ± 0.01 |
| TH-A₂₀ | 1.09 ± 0.07 | 0.95 ± 0.05 | TH-B₂₀ | 1.12 ± 0.09 | 0.92 ± 0.02 |
| TH-A₃₀ | 2.60 ± 0.17 | 0.90 ± 0.04 | TH-B₃₀ | 2.57 ± 0.16 | 0.89 ± 0.03 |
| TH-A₄₀ | 5.87 ± 0.14 | 0.84 ± 0.02 | TH-B₄₀ | 5.20 ± 0.24 | 0.83 ± 0.02 |
| TH-A₅₀ | 7.23 ± 0.29 | 0.82 ± 0.02 | TH-B₅₀ | 6.53 ± 0.50 | 0.83 ± 0.02 |

### EXAMPLE 3

Effect of Excipient on Drug Core Properties

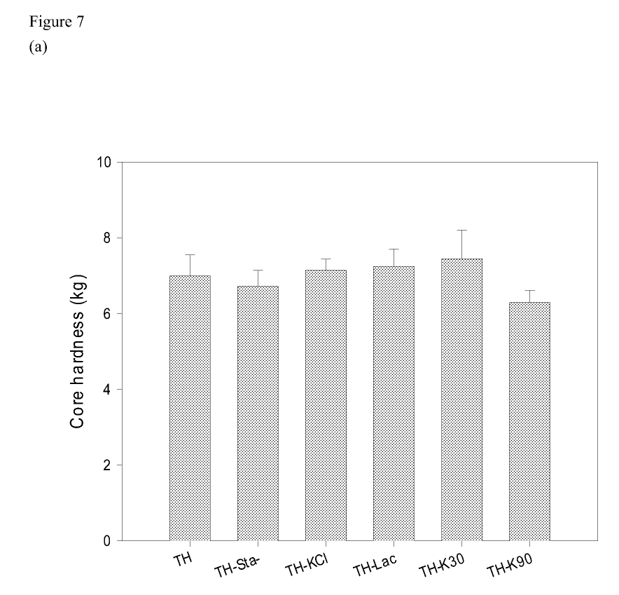

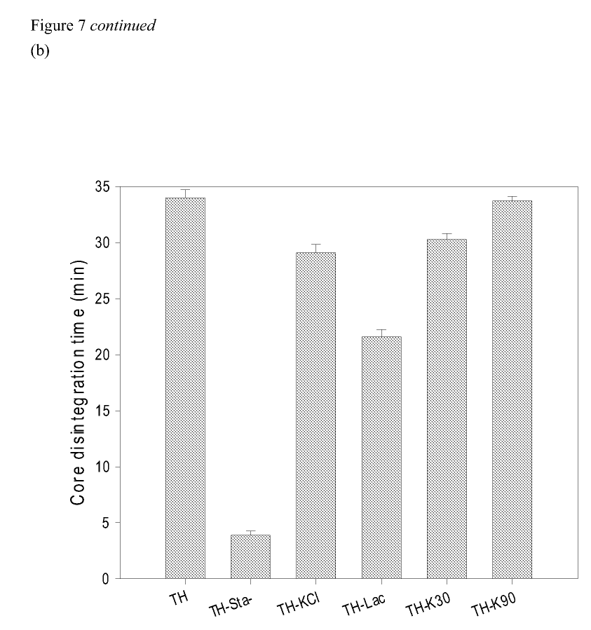

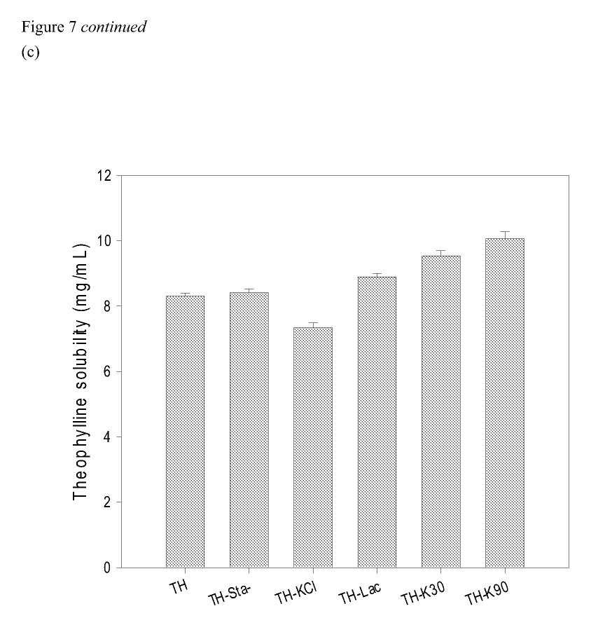

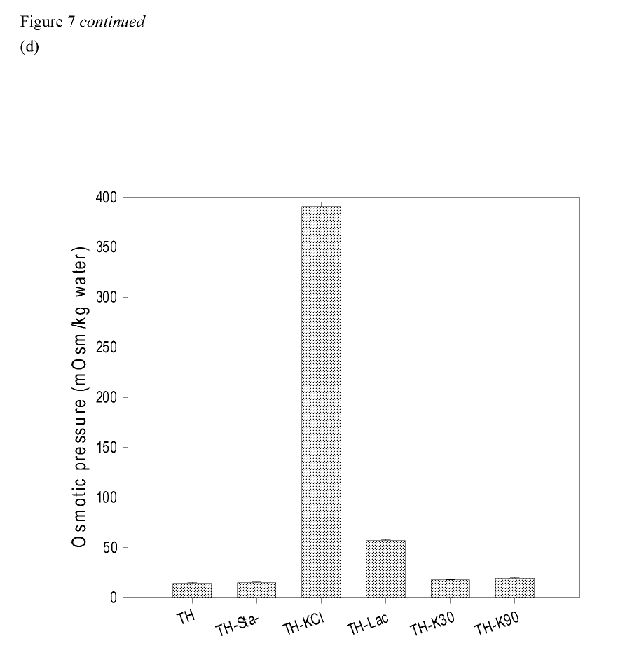

FIG. 7 showed the influences of incorporation of different types of excipient on the performance of theophylline core tablet in terms of its (a) hardness, (b) disintegration time, (c) solubility, and (d) osmotic pressure. Incorporation of different types of excipient influences the property of drug core, wherein lactose was the most adequate excipient. Incorporation of lactose decreased the hardness and the disintegration time of drug core from 7.00±0.55 to 5.73±0.42 kg and from 34.01±0.71 minutes to 6.79±0.08 minutes, respectively, but increased solubility and osmotic pressure from 8.30±0.10 to 9.55±0.14 and from 14.00±1.00 to 127.00±1.00 mOsm/kg, respectively. High water soluble property of lactose enhanced theophylline disintegration, and resulted in increasing theophylline solubility up to 9 times.

### EXAMPLE 4

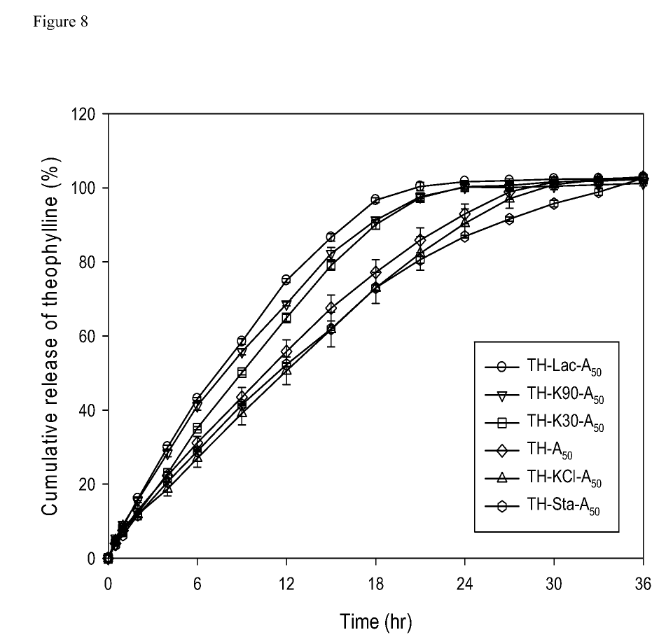

FIG. 8 showed the cumulative release of theophylline from CA_{50%}/PEG_{50%} coated tablets composing different types of excipients. The result showed that the drug continuously releases from the micropore-controlled release tablets for 24-36 hours dependent of the type of excipient in the order of TH-Lac-A₅₀>TH-K90-A₅₀˜TH-K30-A₅₀>TH-A₅₀>TH-KCl-A₅₀TH-Sta-A₅₀. The drug released from the micropore-controlled release tablets composing an excipient of lactose or polyvinylpyrrolidon were faster than the others, wherein lactose was the best excipient to sustain drug release up to 24 hours. Lactose enhanced theophylline solubility and the osmotic pressure of the drug core, which made a positive contribution on drug release. Although potassium chloride produced the highest osmotic pressure in the drug core, however, the salting-out effect following quickly dissolved and leached out of KCl from the drug core result in a slight reduction of drug release. The similar result was observed by using starch as the excipient. Low water solubility of starch hinders drug release from micropore-controlled release tablets.

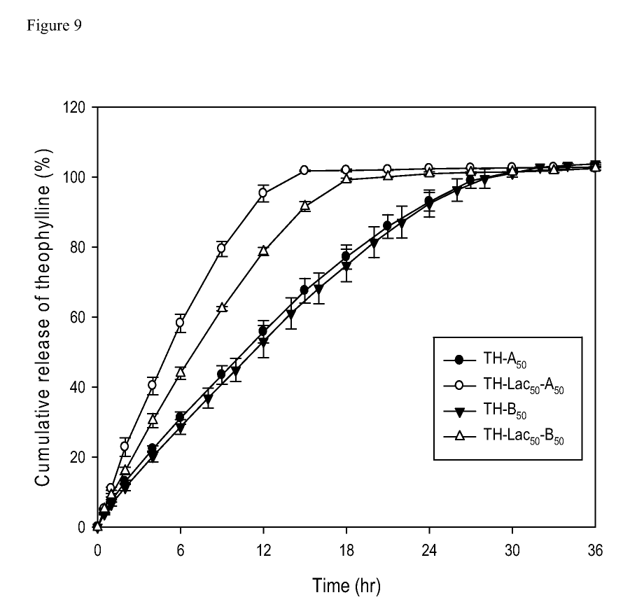

FIG. 9 showed the release of theophylline from micropore-controlled release tablets in the absence or presence of 50% lactose. Lactose as excipient enhanced the drug release rate notably, wherein the drug sustained release from TH-Lac₅₀-A₅₀ and TH-Lac₅₀-B₅₀ micropore-controlled release tablets) up to 18 hours, and the related release rate constants K were 10.68±0.53 and 9.17±0.48% h⁻ⁿ, respectively. The release rate constants K of TH-A₅₀ and TH-B₅₀ micropore-controlled release tablets without lactose were 7.23±0.29 and 6.53±0.50 % h⁻ⁿ, respectively.

### EXAMPLE 5

Pharmacokinetic Study In Vivo

The male rabbits were used as an animal model in this invention. Each rabbit was intravenous injection of theophylline solution, orally administered an uncoated theophylline core tablet (TH-Lac) and a coated micropore-controlled release tablet (TH-Lac-A₅₀), respectively. Blood samples were collected at specific time points, and the plasma concentrations of theophylline were analyzed by high-performance liquid chromatography equipped with a reverse-phase column (Hypersil BDS C18, 250×4.6 mm, 5 μm) and a UV spectrophotometer at a wavelength of 272 nm (Shimadzu SPD-6AV). The mobile phase constituted by acetonitrile and 0.2 M acetate buffer solution (pH 4.5) in the ratio of 6.5:93.5% v/v was applied at a flow rate of 1.0 mL/min.

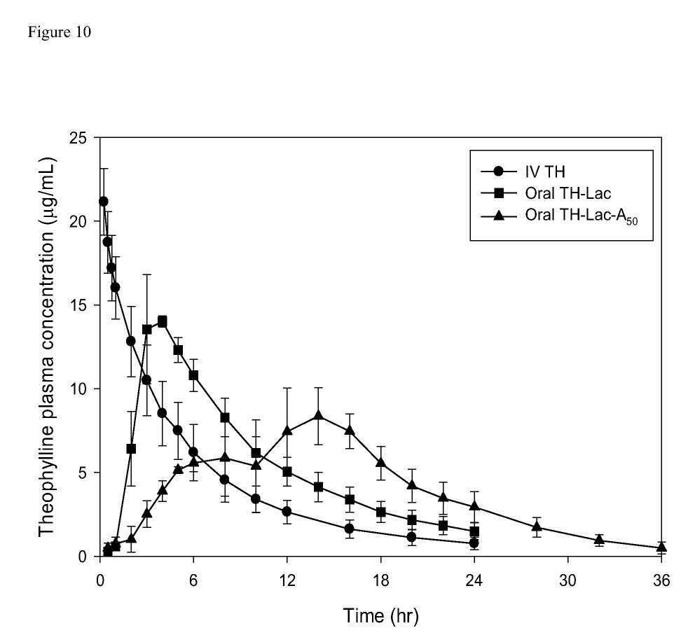

FIG. 10 showed the plasma drug concentration in rabbits after intravenous injection of theophylline solution and oral administrations of uncoated (TH-Lac-A₅₀) and coated theophylline tablets (TH-Lac-A₅₀), respectively. The related pharmacokinetic parameters were listed in Table 3.

TABLE 3
The pharmacokinetic parameters of theophylline after
intravenous injection of theophylline solution, and oral
administration of uncoated tablet (TH-Lac) and coated
tablet (TH-Lac-A₅₀), respectively. (n = 5)

| | | | Oral |
|---|---|---|---|
| Parameter | IV-TH | Oral TH-Lac | TH-Lac-A₅₀ |
| Cₘₐₓ (μg/mL) | 21.15 ± 1.99 | 14.79 ± 1.36 | 8.93 ± 1.91 |
| Tₘₐₓ (hr) | 0.25 ± 0.00 | 3.40 ± 0.55 | 13.60 ± 1.67 |
| AUC∞ (μg · hr/mL) | 116.54 ± 25.88 | 146.89 ± 19.42 | 141.33 ± 27.97 |
| MRT (hr) | 7.70 ± 1.16 | 11.25 ± 1.47 | 15.65 ± 1.72 |

The maximum concentration of drug in plasma (Cₘₐₓ) was 21.15±1.99 μg/mL, which appears at the beginning 0.25 hour followed by quickly reduced after intravenous injection. Oral administration of uncoated tablet (TH-Lac) reduced the maximum concentration to 14.79±1.36 μg/mL. However, oral administration of coated tablet (TH-Lac-A₅₀) even more prominently reduced the maximum concentration to 8.93±1.91 μg/mL, and the time to reach Cmax was delayed from 3.40±0.55 to 13.60±1.67 hr. The mean residence time (MRT) was prolonged from 11.25±1.47 to 15.65±1.72 hr after oral administration of coated tablets, but there was no significant difference in AUC_{∞}. In other words, the micropore-controlled release tablets provided more constant and more sustained drug concentration in plasma than uncoated tablets.

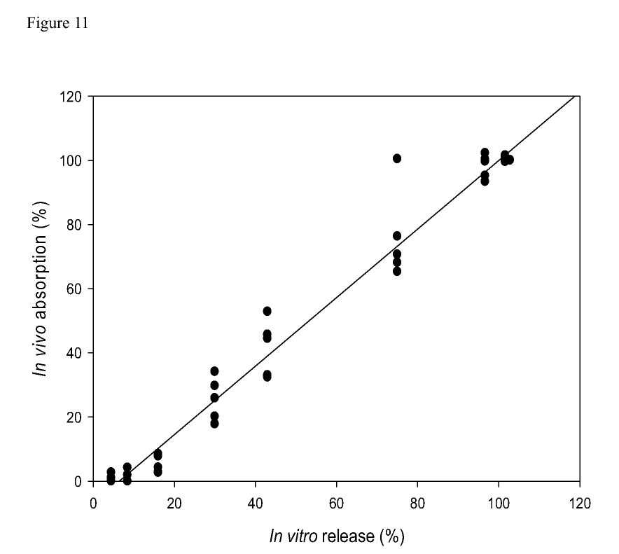

FIG. 11 showed a good correlation between the percentage of drug absorbed in vivo and the percentage of drug released in vitro, and the correlation coefficient was 0.998.

What is claimed is:

1. A microporous film comprising:
(a) a semipermeable polymer; and
(b) a water-soluble polymer,
wherein polymers (a) and (b) form a uniform-blending state through a solvent.

2. The film according to claim 1, wherein the water-soluble polymer is leached out from an aqueous solution to form a microporous film.

3. The film according to claim 1, wherein the distribution density of micropores is proportional to the weight percentage of the water-soluble polymer.

4. The film according to claim 3, wherein the water-soluble polymer comprises from 5 to 50% by weight, and the semipermeable polymer comprises from 50 to 95% by weight.

5. The film according to claim 3, wherein the distribution density of micorepores affects migration velocity from hypertonic solution to hypotonic solution.

6. The film according to claim 1, which is used as a coating material for coating a drug core to form a micropore-controlled release tablet.

7. A method for preparing a micorporous film comprising the steps of:
(a) choosing a suitable formula consisting of a semipermeable polymer, a water-soluble polymer and a solvent;
(b) adding the solvent to completely dissolve the semipermeable polymer and water-soluble polymer to form a polymer blended solution;
(c) controlling temperature of the polymer blended solution and volatile speed of the solvent; and
(d) forming the film when the solvent is evaporated completely.

8. The method of claim 7, wherein the film can further be placed into water to leach the water-soluble polymer to form a microporous film.

9. The method of claim 7, wherein the formula is made by:
(a) molecular weight of the water-soluble polymer,
(b) content ratio of water-soluble polymer and semipermeable polymer;
(c) type of the solvent used to dissolve polymers; and
(d) concentration of polymer blended solution.

10. The method of claim 7, wherein the semipermeable polymer is chosen from cellulose acetate, methyl cellulose acetate (MCA), cellulose diacetate (CDA) or cellulose triacetate(CTA).

11. The method of claim 7, wherein the water-soluble polymer is chosen from polyethylene glycol, polypropylene glycol or poly(ethylene propylene glycol) copolymer.

12. The method of claim 7, wherein the solvent is chosen from ketones, esters, alcohols, alkanes, amides, polar solvents or a mixure of the above.

13. The method of claim 9, wherein the polymer blended solution is from 5 to 15% by concentration.

14. The method of claim 9, wherein the water-soluble polymer is from 1000 to 20000 daltons by molecular weight.

15. The method of claim 9, wherein the water-soluble polymer is from 0 to 50% by weight.

16. The method of claim 9, wherein the semipermeable polymer is from 50 to 95% by weight.

17. A method of preparing a micropore-controlled release tablet, comprising: producing a drug core tablet with drug and excipient; preheating the drug core tablet; coating the drug core tablet with a polymer blended solution, wherein the polymer blended solution comprising: (a) a semipermeable polymer; and (b) a water-soluble polymer, wherein the polymers (a) and (b) form a uniform-blending state through a solvent.

18. The method of claim 17, wherein the preheating temperature is 50° C. with 5 minutes.

19. The method of claim 17, wherein the drug core is composed by sieved drug powder and excipient, which is mixed by geometric dilution method.

20. The method of claim 17, wherein releasing of drug is achieved by the micropores.

21. The method of claim 17, wherein the distribution density of micropores is proportional to the weight percentage of the water-soluble polymer.

22. The method of claim 17, wherein the distribution density of micropores affect the release rate and the release time of drug.
